(19) United States
(12) Patent Application Publication
(10) Pub. No.: US 2007/0117974 A1
(43) Pub. Date: May 24, 2007

(54) One-pot processes for preparing prednisolone derivatives

(75) Inventors: Minghua Ying, Xianju City (CN); Dingwei Wu, Xianju City (CN); Hongpeng Chen, Xianju City (CN)

Correspondence Address: SEED INTELLECTUAL PROPERTY LAW GROUP PLLC, 701 FIFTH AVE, SUITE 5400, SEATTLE, WA, 98104, US

(73) Assignee: Zhejiang Xianju Pharmaceutical Co., Ltd., Xianju City (CN)

(21) Appl. No.: 11/602,479

(22) Filed: Nov. 20, 2006

(30) Foreign Application Priority Data
Jun. 21, 2005 (CN) 200510078712.8

(51) Int. Cl. C07J 71/00

(52) U.S. Cl. 540/70

(57) ABSTRACT

Disclosed is a one-pot process for the preparation of a prednisolone derivative of formula I, comprising reacting the compound of formula II with the compound of formula III and the compound of formula IV The process does not need to separate and purify the intermediate formed, and therefore the process reduces the reaction time and increases the yield of the product compared to the prior art.

### BACKGROUND OF THE INVENTION

1. Technical Field of the Invention

The present invention relates to a process for preparing a prednisolone derivative of formula I, especially to a one-pot process for the preparation of prednisolone derivatives using 16α-hydroxyprednisolone, an anhydride and an aldehyde as starting materials.

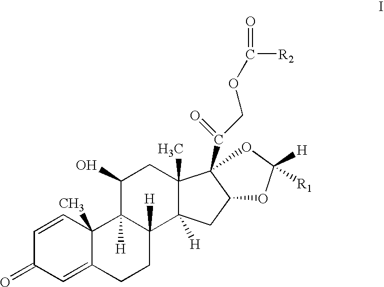

2. Description of Prior Art

Many prednisolone derivatives have been known to be of anti-inflammatory activities, and used for the treatment of skin diseases, respiratory diseases, inflammatory diseases of intestinal tract, allergic rhinitis, conjunctivitis and the like.

Ciclesonide, a compound with the chemical name (22,R)-pregna-1,4-diene-3,20-dione-16α,17-[(cyclohexylmethylene)-dioxy]-11β-hydroxy-21-(2-methyl-1-oxo-acetone), is an important prednisolon e derivative clinically used for treating asthma, chronic obstructive pulmonary disease and rhinitis.

DE 4,129,535 discloses a process for preparing pregna-1,4-diene-3,20-dione-16α,17-acetal-21-esters having a butyl, isopropyl, sec-butyl, cyclohexyl or phenyl radical on the cyclic acetal ring, and whose C₂₁-hydroxyl group is acylated by an acetyl or isobytyryl radical, comprising reacting 16α-hydroxyprednisolone with an anhydride to produce a C-16, C-17 and C-21 esterified intermediate, separating, purifying and then reacting the intermediate with an aldehyde to give the desired product.

In the international application WO 02/38584, a process for preparing a 16α,17-[(cyclohexylmethylene)-bis(oxy)]-11β,21-dihydroxy-pregna-1,4-diene-3,20-dione-21-isobutyrate is disclosed, which comprises reacting a C-21 esterified ketal with cyclohexyl formaldehyde.

The above-mentioned processes are multi-step methods comprising preparing an intermediate from the starting prednisolone, separating, purifying and reacting the intermediate with other reactants to produce the desire product. The methods in the art are less cost-effective for the reason that they need several steps and a long reaction period, and each step for separating or purifying is complicated and may probably cause a loss of the product. In addition, in DE 4,129,535 the yield of the C₂₂-R epimer (the desired prednisolone derivative) in the product is relatively low, and consequently it is difficult to obtain a pure C₂₂-R epimer suitable for pharmaceutical applications.

U.S. Pat. No. 5,733,901 discloses a process for preparing a 16α,17-(22R,S)-[(cyclohexylmethylene)-bis(oxy)]-11β,21-dihydroxy-pregna-1,4-diene-3,20-dione, comprising reacting 16α-hydroxyprednisolone with cyclohexyl formaldehyde.

All patents or patent applications mentioned above are explicitly incorporated herein by reference in their entireties.

### SUMMARY OF THE INVENTION

According to an aspect of the present invention, there is provided a one-pot process for the preparation of a prednisolone derivative of formula I, 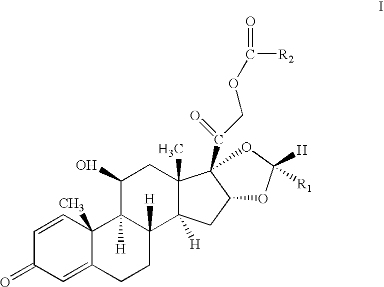
in which
R₁ represents alkyl, alkenyl, alkynyl, cycloalkyl or aryl; and
R₂ represents alkyl, alkenyl or alkynyl,
comprising reacting the compound of formula II, 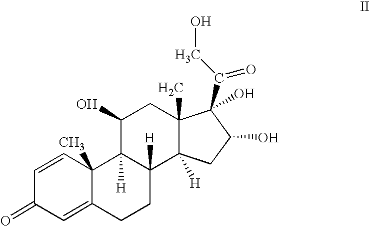
with a compound of formula III, 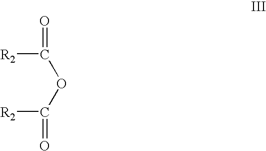
in which R₂ is as previously defined,
and a compound of formula IV, 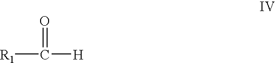
in which R₁ is as previously defined.

In a preferred embodiment of the invention, R₁ represents C₁-C₈ alkyl, C₂-C₈ alkenyl, C₂-C₈ alkynyl, C₃-C₈ cycloalkyl or C₅-C₂₀ aryl.

In a further preferred embodiment of the invention, R₁ is n-butyl, isobutyl, cyclohexyl or phenyl.

In another preferred embodiment of the invention, R₂ represents C₁-C₈ alkyl, C₂-C₈ alkenyl or C₂-C₈ alkynyl.

In a further preferred embodiment of the invention, R₂ is methyl or isopropyl.

The process of the invention can be carried out with or without a solvent. Preferably, the process is carried out in a polar organic solvent.

In some preferred embodiments of the invention, the process is carried out at the presence of a protonic acid catalyst. Preferably, a molar ratio of the catalyst used to the compound of formula II is in the range of 1-10:1, and more preferably in the range of 14:1.

In other preferred embodiments of the invention, the process is carried out at the temperature of 0-50° C.

In the process of the invention, it is unnecessary to separate and purify intermediates formed, and therefore the process is simplified, and the loss of the product, the reaction period and the cost are greatly reduced.

### DETAILED DESCRIPTION OF THE INVENTION

As described above, the present invention provides a one-pot process for the preparation of a prednisolone derivative of formula I, 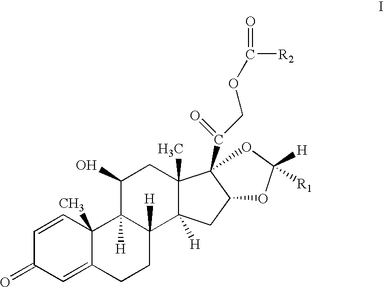
in which
R₁ represents alkyl, alkenyl, alkynyl, cycloalkyl or aryl, and
R₂ represents alkyl, alkenyl or alkynyl,
comprising reacting the compound of formula II, 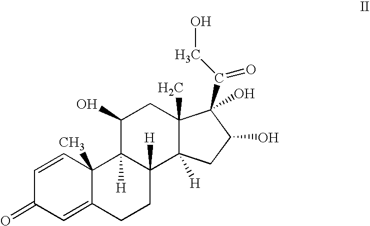
with a compound of formula III, 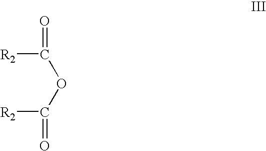
in which R₂ is as previously defined,
and a compound of formula IV, 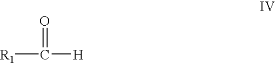
in which R₁ is as previously defined.

The term “alkyl”, as used herein, generally refers to a linear or branched saturated aliphatic radical, preferably refers to C₁-C₈ alkyl, and more preferably refers to C₁-C₆ alkyl, such as methyl, ethyl, n-propyl, isopropyl, n-butyl, isobutyl, t-butyl and the like.

The term “alkenyl”, as used herein, generally refers to a linear or branched unsaturated aliphatic radical having one or more C═C double bonds, preferably refers to C₂-C₈ alkenyl, and more preferably refers to C₂-C₆ alkenyl, such as vinyl, propenyl, allyl, 1-butenyl, 2-butenyl and the like.

The term “alkynyl”, as used herein, generally refers to a linear or branched unsaturated aliphatic radical having one or more C≡C triple bonds, preferably refers to C₂-C₈ alkynyl, and more preferably refers to C₂-C₆ alkynyl, such as ethynyl, 1-propinyl, 2-butynyl, 3-butynyl and the like.

The term “cycloalkyl”, as used herein, generally refers to a saturated alicyclic radical, preferably refers to C₃-C₈ cycloalkyl, and more preferably refers to C₃-C₆ cycloalkyl, such as cyclopropyl, cyclobutyl, cyclopentyl and cyclohexyl.

The term “acyl”, as used herein, generally refers to a univalent radical derived from an aromatic hydrocarbon, preferably refers to C₅-C₂₀ acyl, such as phenyl, naphthyl and the like, and more preferably refers to phenyl.

The term “one-pot process”, as used herein, generally refers to a process for preparing a desired product, comprising simultaneously or successively adding all reactants into a reactor to have them react together, in which no separation and/or purification of the intermediate formed is needed before the product is produced.

The term “intermediate(s)”, as used herein, refers to a resultant of the reaction between any two of the three compounds of formulae II, III and IV, which then reacts with the unreacted compound to form a compound of formula I, or a resultant of the reaction of the three compounds of formulae II, III and IV, which then can be converted into a compound of formula I.

In a particular embodiment of the invention, the intermediate comprises C-16, C-17 and/or C-21 esterified derivatives of prednisolone.

In a preferred embodiment of the invention, R₁ represents C₁-C₈ alkyl, C₂-C₈ alkenyl, C₂-C₈ alkynyl, C₃-C₈ cycloalkyl or C₅-C₂₀ aryl.

In another preferred embodiment of the invention, R₂ represents C₁-C8 alkyl, C₂-C₈ alkenyl or C₂-C₈ alkynyl.

In a further preferred embodiment of the invention, R₁ represents C₁-C8 alkyl, C₂-C₈ alkenyl, C₂-C₈ alkynyl, C₃-C8 cycloalkyl or C₅-C₂₀ aryl, and R₂ represents C₁-C₈ alkyl, C₂-C₈ alkenyl or C₂-C₈ alkynyl.

In a still further preferred embodiment of the invention, R₁ is n-butyl, isobutyl, cyclohexyl or phenyl and R₂ is methyl or isopropyl.

In some preferred embodiments of the invention,. the reactants of the process, i.e. the compounds of formula II, III and IV, are added simultaneously.

In other preferred embodiments of the invention, the reactants of the process are added successively, in which there is no specific requirement for the sequence of addition of the reactants. For example, the reactants can be added in the sequence of II→III→IV (i.e. the compound of formula II followed by the compound of formula III, and then the compound of formula IV), II→IV→III, IV→II→III, IV→III→II, III→II→IV or III→IV→II. More preferably, the compound of formula III and the compound of formula IV are added successively into a solution of the compound of formula I.

Preferably, a molar ratio of the three reactants used is in the range of formula II: formula III: formula IV=1:1-15:1-10, and more preferably is in the range of 1:4-8:2-6. Most preferably the molar ratio of the reactants is about 1:6:4.

In a particularly preferred embodiment of the invention, the reactants are added in the sequence of formula II formula III formula IV, and the molar ratio of the reactants is in the range of formula II: formula III: formula IV=1:4-8:2-6.

In a preferred embodiment of the invention, the process of the invention is carried out without an additional solvent. In this case, it should be appreciated that at least one of the reactants, such as the anhydride or the aldehyde, also serves as a solvent.

In another preferred embodiment of the invention, the process of the invention is carried out at the presence of a polar organic solvent including, but not limited to, ethers, such as ethyl ether, dioxane, dipropyl ether and dibutyl ether; esters, such as ethyl acetate and methyl acetate; halogenated hydrocarbons, such as methylene chloride and chloroform; nitrated hydrocarbons, such as nitromethane, 2-nitropropane and 1-nitropropane.

In a further preferred embodiment of the invention, the solvent used may be selected from the group consisting of dioxane, methylene chloride, chloroform, nitromethane and ethyl acetate, and more preferably the solvent is dioxane.

In some preferred embodiments of the invention, the process is carried out at the presence of a protonic acid catalyst including, but not limited to, hydrochloric acid, sulfuric acid, perchloric acid, methanesulfonic acid, toluene-p-sulfonic acid and tetrafluoroboric acid. Preferably, a molar ratio of the catalyst used to the compound of formula II is in the range of 1-10:1, and more preferably in the range of 1-4:1. Most preferably, the molar ratio of the catalyst to the compound of formula II is about 3.8:1.

In a further preferred embodiment of the invention, the catalyst used may be selected from the group consisting of hydrochloric acid, tetrafluoroboric acid, methanesulfonic acid and perchloric acid. More preferably, the catalyst is a 35-70% perchloric acid, and further more preferably the catalyst is a 60-70% perchloric acid. Most preferably, the catalyst is a 70% perchloric acid.

In another preferred embodiment of the invention, the process is carried out at a temperature in the range of 0-50° C., more preferably in the range of 0-30° C., and further more preferably in the range of 20-30° C. In the process of the invention, if the reaction temperature is too low (such as lower than 0° C.), the dissolution of the reactants and the intermediated formed will be deteriorated. And if the reaction temperature is too high (such as higher than 50° C.), the C-21 ester of the compound of formula I will be decomposed, which will cause a reduction of the yield of the desired product.

In a particularly preferred embodiment of the invention, R₁ is n-butyl, isobutyl, cyclohexyl or phenyl and R₂ is methyl or isopropyl, and the process is carried out at the presence of dioxane and a catalyst of 60-70% perchloric acid, and at the temperature of 20-30° C., in which the molar ratio of the compound of formula II, the compound of formula III and the compound of formula IV is 1:4-8:2-6, and the molar ratio of the catalyst to the compound of formula II is 1-4:1.

In a most preferred embodiment of the invention, R₁ is n-butyl, isobutyl, cyclohexyl or phenyl and R₂ is methyl or isopropyl, and the process is carried out at the presence of dioxane and a catalyst of 70% perchloric acid, and at the temperature of 20-30° C., in which the molar ratio of the compound of formula II, the compound of formula III and the compound of formula IV is about 1:6:4, and the molar ratio of the catalyst to the compound of formula II is about 3.8:1.

In a preferred embodiment of the invention, the process is carried out for about 2-10 hours, and more preferably for about 5-8 hours.

In a preferred embodiment of the invention, the process further comprises the steps for separating and purifying the desired product. The steps may be performed using conventional separating and purifying techniques, such as preparative High Performance Liquid Chromatography (HPLC) and fractional crystallization.

Besides the benefits of shortening the reaction period and reducing the production cost, the process of the invention has an advantage that the ratio of the C₂₂-R epimer (the desired prednisolone derivative) to the C₂₂-S epimer in the product is increased to R/S≧95/5.

Hereinafter, the invention will be illustrated more in detail by the following examples which show various aspects and advantages of the invention. However, it should be understood that the examples below are non-limiting and are only illustrative of some of the embodiments of the present invention.

### EXAMPLES

### Example 1

To a suspension of 10.0 g of 16α-hydroxyprednisolone (26.6 mmol) in 100 ml of dioxane, was added 8.8 ml of 70% perchloric acid (102.4 mmol) under stirring. 26.5 ml of isobutyric anhydride (159.6 mmol) was then added dropwise within 10 minutes, followed by 12.8 ml of cyclohexyl formaldehyde (106.4 mmol) for 10 minutes. The resultant was stirred under room temperature for 5 hours, neutralized with an aqueous solution of sodium carbonate, and then extracted with ethyl acetate. The organic phase was separated, washed with water, dried in sodium sulfate, and concentrated under vacuum. The residue was recrystallized in ether/petroleum ether to give 11.8 g of the product, yield 82% and R/S=96.5/3.5.

### Example 2

To a suspension of 10.0 g of 16α-hydroxyprednisolone (26.6 mmol) in 100 ml of dioxane, was added 8.8 ml of 70% perchloric acid (102.4 mmol) under stirring. 15.1 ml of acetic anhydride (159.6 mmol) was then added dropwise within 10 minutes, followed by 12.8 ml of cyclohexyl formaldehyde (106.4 mmol) for 10 minutes. The resultant was stirred under room temperature for 7 hours, neutralized with an aqueous solution of sodium carbonate, and then extracted with ethyl acetate. The organic phase was separated, washed with water, dried in sodium sulfate, and concentrated under vacuum. The residue was recrystallized in ether/petroleum ether to give 11.1 g of the product, R/S=96.8/3.2.

### Example 3

To a suspension of 110.0 g of 16α-hydroxyprednisolone (26.6 mmol) in 100 ml of dioxane, was added 8.8 ml of 70% perchloric acid (102.4 mmol) under stirring. 15.1 ml of acetic anhydride (159.6 mmol) was then added dropwise within 10 minutes, followed by 6.0 ml of acetaldehyde (106.4 mmol) for 10 minutes. The resultant was stirred under room temperature for 6 hours, neutralized with an aqueous solution of sodium carbonate, and then extracted with ethyl acetate. The organic phase was separated, washed with water, dried in sodium sulfate, and concentrated under vacuum. The residue was recrystallized in ether/petroleum ether to give 9.8 g of the product, R/S=96.0/4.0.

It should be understood that although the present invention has been specifically disclosed by preferred embodiments and descriptions, modifications and variations to the present invention, without departing from the spirit of the invention, may be made by one of ordinary skill in the art, and that such modifications and variations will fall within the scope of the invention.

1. A one-pot process for the preparation of a prednisolone derivative of formula I, 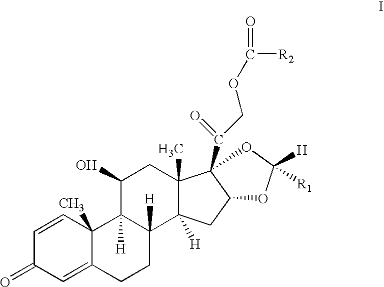
wherein
R₁ represents alkyl, alkenyl, alkynyl, cycloalkyl or aryl; and
R₂ represents alkyl, alkenyl or alkynyl,
comprising reacting the compound of formula II, 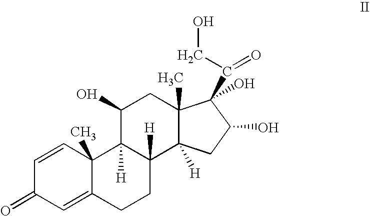
with a compound of formula III, 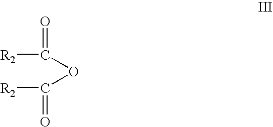
wherein R₂ is as previously defined,
and a compound of formula IV, 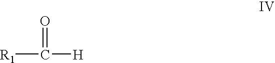
wherein R₁ is as previously defined.

2. The process according to claim 1, wherein R₁ represents C₁-C₈ alkyl, C₂-C₈ alkenyl, C₂-C₈ alkynyl, C₃-C₈ cycloalkyl or C₅-C₂₀ aryl.

3. The process according to claim 2, wherein R₁ is n-butyl, isobutyl, cyclohexyl or phenyl.

4. The process according to claim 1, wherein R₂ represents C₁-C₈ alkyl, C₂-C₈ alkenyl or C₂-C₈ alkynyl.

5. The process according to claim 4, wherein R₂ is methyl or isopropyl.

6. The process according to claim 1, wherein R₁ represents C₁-C₈ alkyl, C₂-C₈ alkenyl, C₂-C₈ alkynyl, C₃-C₈ cycloalkyl or C₅-C₂₀ aryl, and R₂ represents C₁-C₈ alkyl, C₂-C₈ alkenyl or C₂-C₈ alkynyl.

7. The process according to claim 6, wherein R₁ is n-butyl, isobutyl, cyclohexyl or phenyl, and R₂ is methyl or isopropyl.

8. The process according to Claim 1, wherein a molar ratio of the compound of formula II, the compound of formula III and the compound of formula IV is 1:1-15:1-10.

9. The process according to claim 8, wherein the molar ratio is 1:4-8:2-6.

10. The process according to claim 1, wherein the process is carried out at the temperature of 0-50° C.

11. The process according to claim 10, wherein the process is carried out at the temperature of 20-30° C.

12. The process according to claim 1, wherein the process is carried out at the presence of a protonic acid catalyst.

13. The process according to claim 12, wherein the protonic acid catalyst is selected form the group consisting of hydrochloric acid, sulfuric acid, perchloric acid, methanesulfonic acid, toluene-p-sulfonic acid and tetrafluoroboric acid.

14. The process according to claim 13, wherein the protonic acid catalyst is a 35-70% perchloric acid.

15. The process according to claim 14, wherein the protonic acid catalyst is a 60-70% perchloric acid.

16. The process according to claim 12, wherein a molar ratio of the protonic acid catalyst to the compound of formula II is 1-10:1.

17. The process according to claim 16, wherein the molar ratio of the protonic acid catalyst to the compound of formula II is 1-4:1.

18. The process according to claim 1, wherein the process is carried out at the presence of a polar organic solvent selected from the group consisting of dioxane, methylene chloride, chloroform, nitromethane and ethyl acetate.

19. A one-pot process for the preparation of a prednisolone derivative of formula I, 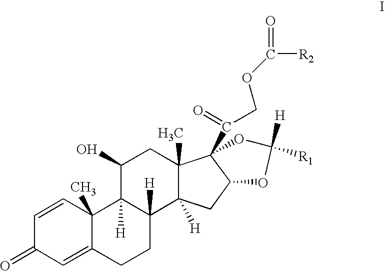
wherein
R₁ is n-butyl, isobutyl, cyclohexyl or phenyl; and
R₂ is methyl or isopropyl,
comprising reacting the compound of formula II, 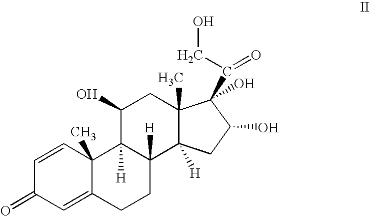
with a compound of formula III, 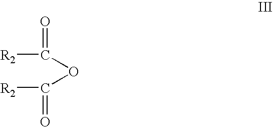
wherein R₂ is as previously defined,
and a compound of formula IV, 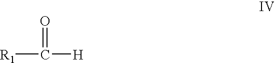
wherein R₁ is as previously defined,
at the presence of dioxane and a catalyst of 60-70% perchloric acid, and at the temperature of 20-30° C.,
and wherein a molar ratio of the compound of formula II, the compound of formula III and the compound of formula IV is 1:4-8:2-6, and a molar ratio of the catalyst to the compound of formula II is 1-4:1.

20. The process according to claim 19, wherein the catalyst is a 70% perchloric acid, and the molar ratio of the compound of formula II, the compound of formula III and the compound of formula IV is about 1:6:4, and the molar ratio of the catalyst to the compound of formula II is about 3.8:1.
